(19) United States
(12) Patent Application Publication
(10) Pub. No.: US 2024/0116686 A1
(43) Pub. Date: Apr. 11, 2024

(54) STORAGE CONTAINER FOR FREEZING, THAWING, AND SHIPPING

(71) Applicant: ENTEGRIS, INC., Billerica, MA (US)

(72) Inventors: Nicholas Coscia, Lexington, MA (US); Jacob Peabody, Roxbury, MA (US); Brian M. Fidler, Nashua, NH (US); Aaron L. Ersando, Windsor, CT (US); Robert M. Shydo, JR., Hampstead, NH (US)

(21) Appl. No.: 18/378,110

(22) Filed: Oct. 9, 2023

Related U.S. Application Data
(60) Provisional application No. 63/414,818, filed on Oct. 10, 2022.

(51) Int. Cl. B65D 55/10; B65D 43/02; B65D 81/133

(52) U.S. Cl. CPC B65D 55/10; B65D 43/0233; B65D 81/133; B65D 2543/00277; B65D 2581/055

(57) ABSTRACT

A storage container that includes a housing having a bottom surface, two side walls, a front wall, and a back wall that form an interior space in the housing. The storage container also includes a lid configured to enclose the interior space. The two side walls of the housing include a rim portion and the lid includes a rail portion configured to provide a non-destructive and deformable locking and unlocking engagement with the rim portion. The back wall of the housing includes a first segment and a second segment, the second segment being separated a distance longitudinally from the first segment and connected to the first segment by a secondary bottom surface such that the second section of the interior space has a greater volume than the first section of the interior space.

### FIELD

This disclosure is directed to storage containers, such as bag holders, for supporting bags during any of the freezing, thawing, or frozen shipping processes, particularly storage containers that protect the bag during use, transport, or shipping and have a design that facilitates the use and reuse of the storage container.

### BACKGROUND

Chemical or biological processes can be carried out, or their results contained in bags such as bags containing pharmaceutical or biological fluids, bioprocess bags, and the like. Process materials or materials resulting from the process may need to be frozen or otherwise kept at low temperature. Bags may need to be supported through cooling or freezing processes to ensure the integrity of the bag or maintenance of a desirable shape. Holders may be used to support and shape the bags before, during, or after the cooling or freezing process during transport or shipping.

### SUMMARY

This disclosure is directed to storage containers, such as bag holders, for supporting bags during any of the freezing, thawing, or frozen shipping processes, particularly storage containers that protect the bag during use, transport, or shipping and have a design that facilitates the use and reuse of the storage container.

Use of bag holders for plate freezing processes can fix those bags such that they provide a shape having a good contact surface for freezing, and maintain this shape as the material contained in the bag is frozen, for example, when aqueous solutions expand during freezing. Typical bag holders, however, have a generic shape and construction that is not suitable for certain types of bioprocess bags.

For example, particular type of bags (e.g., made of fluoropolymer or the like) are commonly used for e.g., plate freezing and thawing applications, while typical bag holders are designed and built around the need of polyethylene (PE) bags that have more elasticity than the fluoropolymer bags. This may result in the fluoropolymer bags experiencing multiple failures in the typical bag holders, which may deter users from using the fluoropolymer bags in the process. Additionally, some types of bags have different shapes and designs than the more general PE bags by having angled edges connected to a bottom edge of the bag. In view of the different shape of such bags, the typical bag holders are not suitable for adequately securing and protecting the bag, which can result in failure of the bag during the freezing, thawing, storage, or transport processes. Furthermore, most bag holders are designed for single-use. For example, the bag holders include plastic or metal tabs that are permanently bent to form the housing of the bag holder or to secure the lid to the housing. After multiple-uses, however, the plastic or metal tabs commonly break and create sharp edges that pose a puncture or cut hazard. The typical bag holder is also made from plastic or other material, which is not only expensive to manufacture, such material is not typically suitable for multiple uses and cleanings. As such, the typical bag holders have at least the following deficiencies: connectors or tubing connected to the bag are incorrectly placed in the bag holder, the fluoropolymer bag has a different shape which can cause the front edge of the lid to catch the foam and bag during the sliding action to close the bag holder, are expensive to manufacture due to the machined plastic design, have a single-use design, and due to the sliding action of the lid, it is difficult to enclose the interior space having the foam and materials bag.

Embodiments disclosed herein can provide solutions to the above problems by e.g., making the bag holder more robust, facilitating the use or reuse of the bag holder, and protecting the bags during closing, handling, transport, or shipping.

Embodiments disclosed herein can also provide flexibility to modify the bag support configuration within the bag holder to improve the performance of the bag, does not use metal bend tabs that are permanently bent which are used with typical bag holders, make opening and closing the bag holder easier without reducing the effectiveness of the bag holder, and provide security features to the bag holders.

In an embodiment, a storage container for pharmaceutical or biological materials includes a housing, and a lid. The housing includes a bottom surface, two side walls, a front wall, and a back wall that form an interior space in the housing. The interior space has a first section and a second section that is provided above the first section. The lid is configured to enclose the interior space. The two side walls of the housing include a rim portion at a top portion of the two side walls and the lid includes a rail portion configured to provide a non-destructive and deformable locking and unlocking engagement with the rim portion of the two side walls. The back wall of the housing includes a first segment and a second segment provided above the first segment, the second segment being separated a distance longitudinally from the first segment and connected to the first segment by a secondary bottom surface such that the second section of the interior space has a greater volume than the first section of the interior space.

In an embodiment, the rail portion of the lid is also configured to provide a sliding engagement with the rim portion of the two side walls.

In an embodiment, the front wall includes an indentation portion such that the indention portion protrudes into the interior space of the housing.

In an embodiment, the indentation portion includes a handle portion configured for lifting, pushing, or pulling the storage container.

In an embodiment, a first foam portion is provided in the interior space and configured to conform to a shape of a pharmaceutical or biological materials bag. A second foam portion is also provided in the interior space adjacent to the first foam portion and configured to receive any tubing or connections attached to the pharmaceutical or biological materials bag. The second foam portion has a lower rigidity than the first foam portion.

In an embodiment, the first foam portion includes a first portion and a second portion separate from the first portion. The first portion of the first foam portion is configured to fit in the first section of the interior space and the second portion of the first foam portion is configured to fit in the second section of the interior space above the first portion of the first foam portion.

In an embodiment, the shape of the pharmaceutical or biological materials bag includes angled edges connected to a bottom edge of the pharmaceutical or biological materials bag.

In an embodiment, the rim portion of the two side walls and the rail portion of the lid include openings configured to align the lid with the housing and configured to receive a securing device for securing the lid to the housing.

In an embodiment, the securing device include at least one of a zip tie, a securement pin or lock, or a push rivet.

In an embodiment, the lid further includes a back rail portion configured to engage the back wall of the housing to align the openings in the rim portion of the two side walls and the rail portion of the lid.

In an embodiment, the lid has one end pivotally connected to the housing such that the rail portion is configured to provide the non-destructive and deformable locking and unlocking engagement with the rim portion of the two side walls.

In an embodiment, the lid is removably attachable to the housing.

In an embodiment, the rail portion of the lid includes a downwardly open groove configured to engage the rim portion of the two side walls.

In an embodiment, the rail portion further includes a handhold portion connected to the downwardly open groove for providing the non-destructive and deformable unlocking engagement between the rim portion and the rail portion to remove the lid from the storage container.

In an embodiment, the housing and lid are formed completely from metal.

In an embodiment, the bottom surface, the two side walls, the front wall, and the back wall are connected by at least one of a self-clinching fastener, pin, screw, nut, rivet, or welding.

In an embodiment, the first foam portion further includes a partial fill foam portion configured to displace a volume of the pharmaceutical or biological materials bag.

In an embodiment, a storage container for pharmaceutical or biological materials, includes a housing, a lid, a first foam portion, and a second foam portion. The housing includes a bottom surface, two side walls, a front wall, and a back wall that form an interior space in the housing. The interior space has a first section and a second section that is provided above the first section. The lid is configured to enclose the interior space. The first foam portion is provided in the interior space and configured to conform to a shape of a pharmaceutical or biological materials bag. The second foam portion is provided in the interior space adjacent to the first foam portion and configured to receive any of tubing or connections attached to the pharmaceutical or biological materials bag. The two side walls of the housing include a rim portion at a top portion of the two side walls and the lid includes a rail portion configured to provide a non-destructive and deformable locking and unlocking engagement with the rim portion of the two side walls. The back wall of the housing includes a first segment and a second segment provided above the first segment. The second segment is separated a distance longitudinally from the first segment and connected to the first segment by a secondary bottom surface such that the second section of the interior space has a greater volume than the first section of the interior space. The front wall includes an indentation portion such that the indention portion protrudes into the interior space of the housing and into the second foam portion. The indentation portion includes a handle portion configured for lifting, pushing, or pulling the storage container.

In an embodiment, a method for storing a pharmaceutical or biological materials bag includes providing a first portion of a first foam portion in a first section of an interior space of a housing of a storage container. The housing includes a bottom surface, two side walls, a front wall, and a back wall that form the interior space in the housing. The method further includes providing a first portion of a second foam portion in the first section of the interior space adjacent the first portion of the first foam portion, placing the pharmaceutical or biological materials bag over the first portion of the first foam portion and the first portion of the second foam portion such that the pharmaceutical or biological materials bag is provided on the first foam portion and tubing and connectors connected to the pharmaceutical or biological materials bag are provided on the second foam portion, and providing a second portion of the first foam portion and a second portion of the second foam portion in a second section of the interior space such that the pharmaceutical or biological materials bag and the tubing and the connectors are between the first portion and the second portion of the first foam portion and the second foam portion. The second section is provided above the first section. The method also includes non-destructively deforming a rail portion of the lid to provide a locking engagement of the lid to the housing to enclose the interior space of the housing by engaging the rail portion of the lid with a rim portion at the top portion of the two side walls. The back wall of the housing includes a first segment and a second segment provided above the first segment. The second segment is separated a distance longitudinally from the first segment and connected to the first segment by a secondary bottom surface such that the second section of the interior space has a greater volume than the first section of the interior space.

In an embodiment, the lid is pivotally connected to the housing such that the lid encloses the interior space by pivoting the lid until the non-destructive deforming of the rail portion of the lid is in the locking engagement with the rim portion of the two side walls.

In an embodiment, the method further includes non-destructively deforming the rail portion of the lid to provide an unlocking engagement of the lid to expose the interior space, without damaging the lid.

The details of one or more embodiments of the disclosure are set forth in the accompanying drawings and the description, and from the claims.

### BRIEF DESCRIPTION OF DRAWINGS

References are made to the accompanying drawings that form a part of this disclosure, and which illustrate embodiments in which the systems and methods described in this specification can be practiced.

FIG. 1A shows a storage container with a lid removed according to an embodiment.

FIG. 1B shows the storage container of FIG. 1A with the lid enclosing an interior space.

FIG. 1C shows a cross-sectional view of the storage container of FIG. 1B.

FIG. 1D shows an enlarged end view of a back wall of the storage container of FIG. 1B.

FIG. 1E shows an enlarged end view of a front wall of the storage container of FIG. 1B.

FIG. 1F shows a cross-sectional view of the cavity and handle of the storage container of FIG. 1E.

FIGS. 2A-2F shows an assembly of a storage container according to an embodiment.

FIG. 3 shows a storage container according to another embodiment.

FIGS. 4A-4B show a storage container according to yet another embodiment.

FIGS. 5A-5B show a storage container according to yet another embodiment.

FIGS. 6A-6B show an end view of the back wall and lid of the storage container according to an embodiment.

Like reference numbers represent like parts throughout.

### DETAILED DESCRIPTION

This disclosure is directed to storage containers, such as bag holders, for supporting bags during any of the freezing, thawing, or frozen shipping processes, particularly storage containers that protect the bag during use, transport, or shipping and have a design that facilitates the use and reuse of the storage container.

As defined herein, the phrase “a plate freezing process” may refers to a process of freezing object(s) (e.g., bag holder(s) or the like) via e.g., plate freezer(s). A plate freezer can be e.g., a hollow plate that has liquid refrigerant flowing through it. Objects can be placed between two plates and evenly cooled to freezing temperatures. The plate freezers can be ideal for freezing flat objects or brick-shaped packaged objects.

As defined herein, the phrase “a blast freezing process” may refer to a process of freezing object(s) (e.g., bag holder(s) or the like) via e.g., blast freezer(s). A blast freezer can be e.g., a mechanical system generally used to freeze products in bulk on pallets. It will be appreciated that the phrase “a thawing process” may refer to a process designed to thaw objects instead of freezing objects.

As defined herein, the phrase “non-destructive” may refer to a process in which the structure is not broken or unusable after iterative uses, such that the structure can be reusable. For example, a typical bag holder can include a metal or plastic tab(s) for securing a lid to the housing by permanently bending the tab(s). After a set number of permanent bendings of the tab, e.g., more than 2 bend, or 5 bends, or more beyond the elastic limits of the material, e.g., permanently bent, the tab will break and not be able to secure the lid, e.g., fracture of the tab. In the present disclosure, the non-destructive process may refer to an elastic deformation of the material such that the material is not broken or unusable after iterative uses, especially in a clean room environment. It is appreciated that the term “bent” as used herein refers to a permanent bending, e.g., beyond the elastic limits, such that the material maintains the shape after the bending, e.g., no elastic restoring force.

As defined herein, the term “substantially” may refer to a configuration that is similar to or within a tolerance or variation that is at least 5% of any stated value.

Embodiments disclosed herein can provide a robust storage container for pharmaceutical or biological materials designed for any of plate freezing, blast freezing, robust frozen shipping, or thawing technologies, especially when used in a clean room environment.

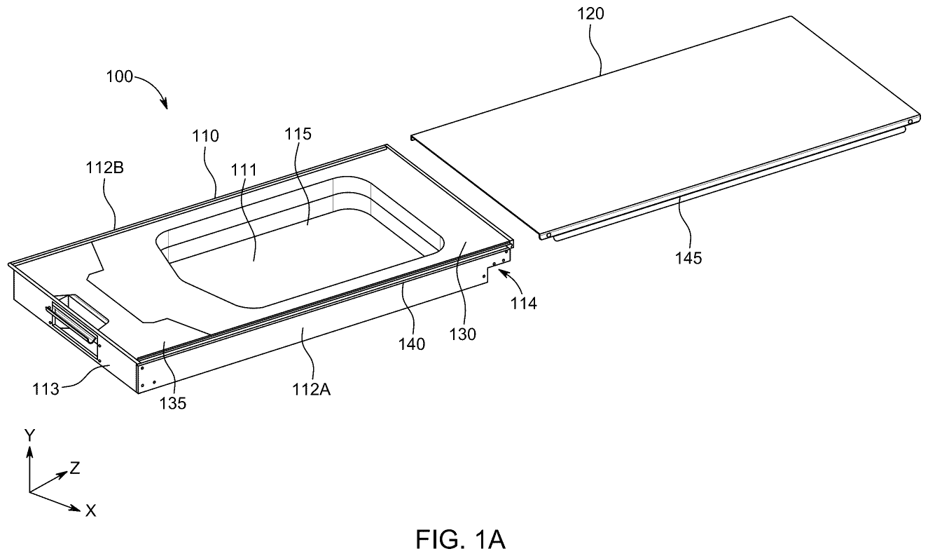

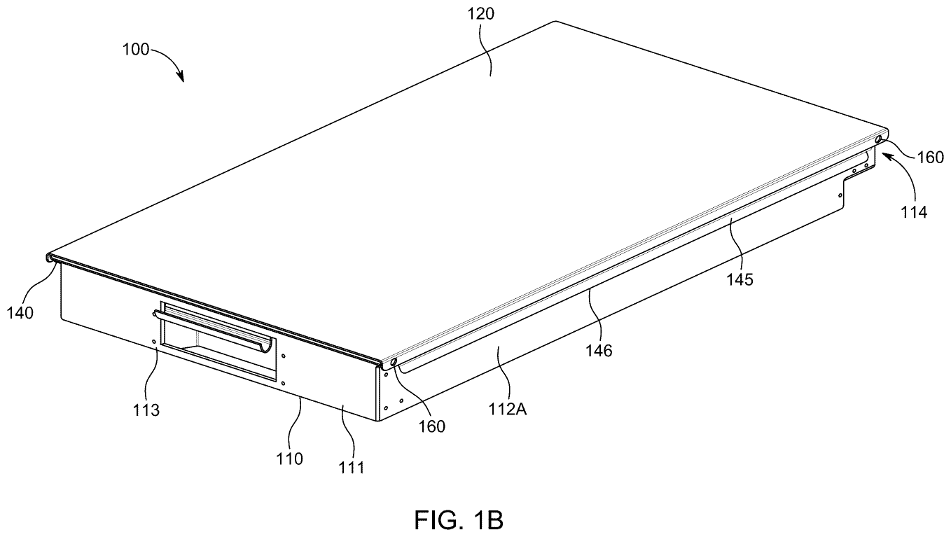

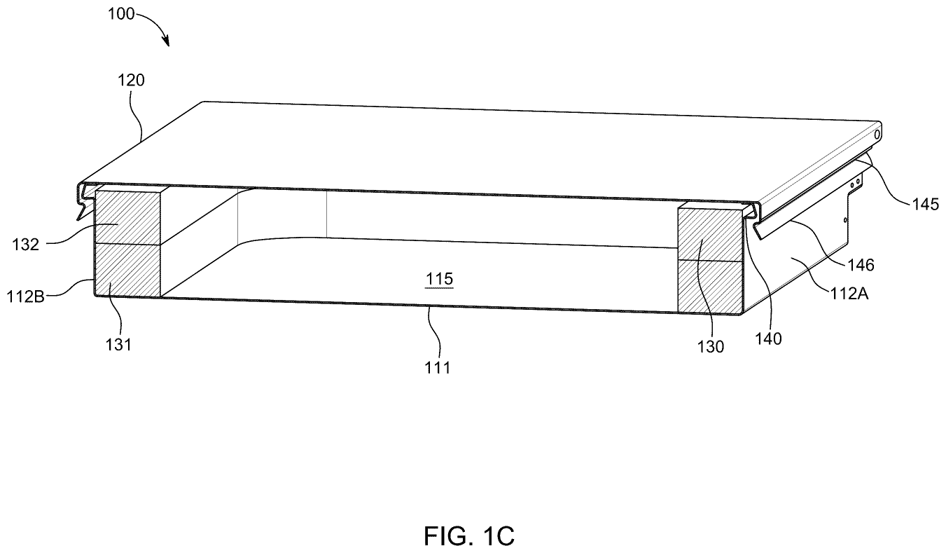

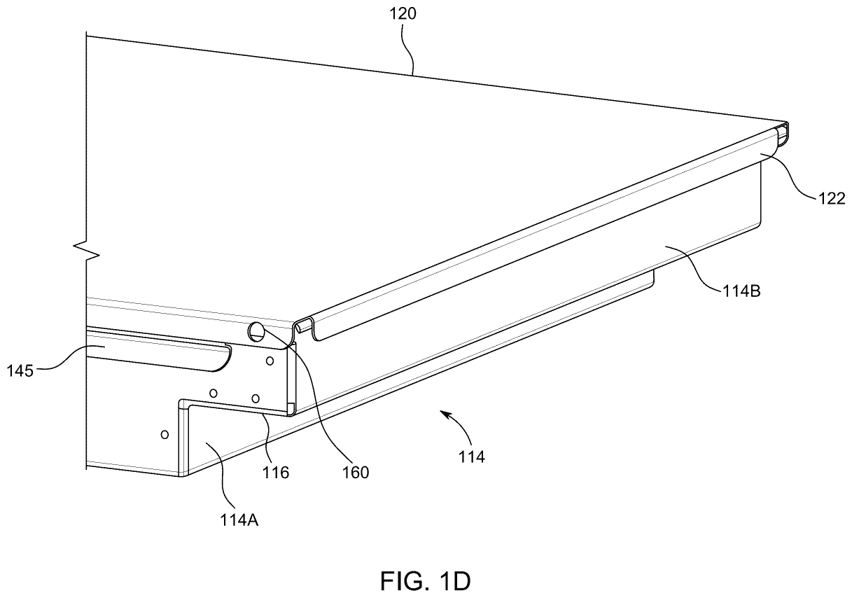

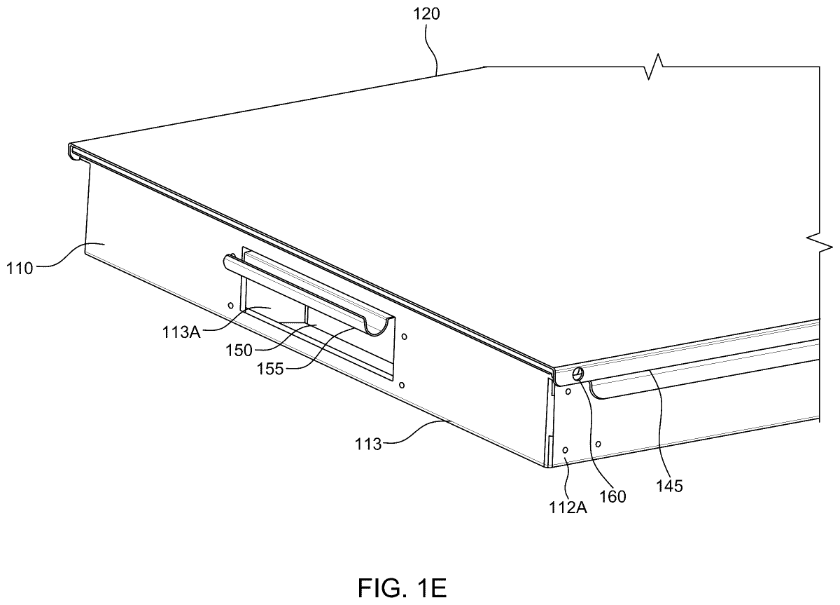

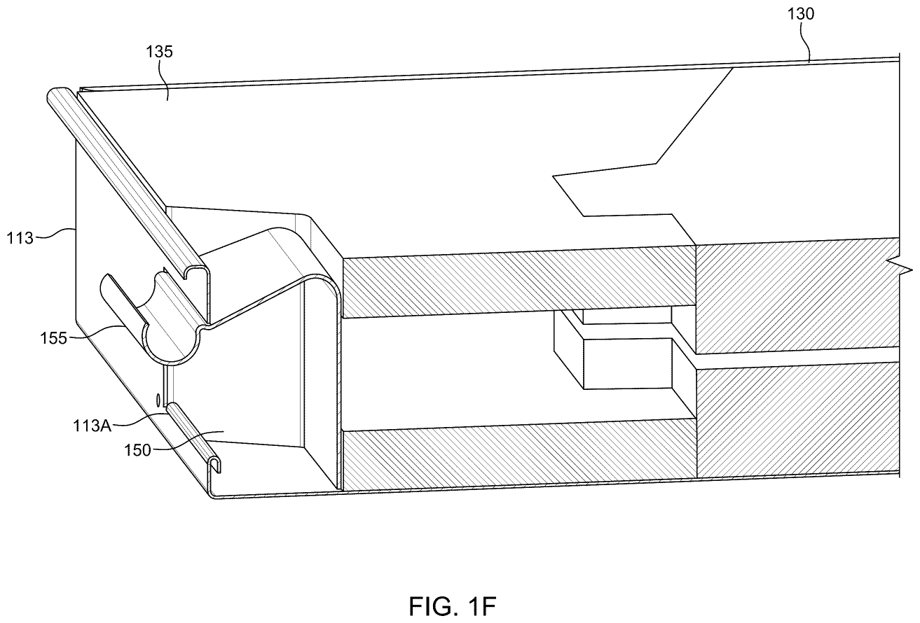

FIG. 1A illustrates a perspective view of a storage container, e.g., a bag holder, 100, according to an embodiment with the lid removed. FIG. 1B is the storage container 100 of FIG. 1A viewed with the lid attached to enclose the interior space, according to an embodiment. FIG. 1C illustrates a cross-sectional view of the storage container 100 of FIG. 1B. FIG. 1D illustrates an enlarged end view of the back wall of the storage container 100 of FIG. 1B. FIG. 1E illustrates an enlarged end view of the front wall of the storage container 100 of FIG. 1B. FIG. 1F illustrates an enlarged cross-sectional view of the cavity and handle of the storage container 100 of FIG. 1E.

As shown in FIGS. 1A and 1B, the storage container 100 has a thickness or height in a vertical direction (Y direction). The storage container 100 has a width in a horizontal or transverse direction (X direction). The storage container 100 has a length in a longitudinal direction (Z direction). As defined herein, the phrase “a thickness direction” may refers to the Y direction, indicating the thickness direction of the storage container 100. The phrase “a width direction” may refers to the X direction, indicating the width direction of the storage container 100. The phrase “a longitudinal direction” may refers to the Z direction, indicating the length direction of the storage container 100.

The storage container 100 includes a housing 110, a lid 120, and a first foam portion 130 and a second foam portion 135. It will be appreciated that the storage container 100 can include at least one foam portion (130, 135).

The housing 110 includes a bottom surface 111, two side walls 112A, 112B, a front wall 113, and a back wall 114 that form an interior space 115 in the housing 110. In an embodiment, the two side walls include a first side 112A and a second side 112B that can be identical to each other. The bottom surface 111, two side walls 112A, 112B, front wall 113, and back wall 114 can be connected together using at least one of self-clinching fasteners, e.g., PEM fasteners, pins, screws, nuts, rivets, or welding at the corners or ends thereof.

FIG. 1C illustrates, in an embodiment, that the two side walls 112A, 112B includes a rim portion 140 at a top portion of the side walls 112A, 112B. In an embodiment, the rim portion 140 includes a bent portion that extends outwardly from the housing and is bent inwardly towards the housing. In an embodiment, the rim portion 140 can have a cross-sectional area that is circular, triangular, rectangular, rhomboidal, or have other geometric designs. The disclosure herein is not intended to limit the scope of the design of the rim portion 140, but rather, provided for the understanding of the disclosure, and the rim portion 140 can be provided with a variety of designs for engaging the lid 120, as further discussed below.

As shown in FIG. 1D, the back wall 114 of the housing 110 includes a first segment 114A and a second segment 114B provided above the first segment 114A. The second segment 114B is separated a distance longitudinally, with respect to the length of the storage container 100, from the first segment 114A. The second segment 114B is connected to the first segment by a secondary bottom surface 116. The bottom surface 111 and the first segment 114A of the back wall 114 form a first section of the interior space. The extension of the secondary bottom surface 116 and the second segment 114B form a second section of the interior space that is above the first section and the second section has a greater volume than the first section. It is appreciated that the extended section of the storage container 100 can also be considered a stepped section or ledge section such that the second section has a greater volume than the first section.

As illustrated in FIG. 1E, the front wall 113 includes an indentation portion 113A that protrudes into the interior space of the housing 110. The indentation portion 113A can be formed as a hole or opening in the front wall 113 and formed as a cavity 150 that protrudes or is positioned within the interior space 115.

FIG. 1F, which is a cross-sectional view of FIG. 1E, illustrates that the cavity 150 seals the interior space 115 along with the front wall 113. The cavity 150 can be provided as a separate element than the storage container 100 or formed during the manufacturing of the storage container 100. If the cavity 150 is provided as a separate element, the cavity 150 can be attached or connected to the storage container 100 using at least one of self-clinching fasteners, e.g., PEM fasteners, pins, screws, nuts, rivets, or welding. The cavity 150 can include a handle portion 155 configured for lifting, pushing, or pulling the storage container 100. In an embodiment, the handle portion 155 can be a continuous portion formed from the cavity 150 that is curved or angled to facilitate the lifting, pushing, or pulling of the storage container 100. It is appreciated that in prior designs of the typical bag holders, the typical bag holders had small openings that made it difficult to hold the bag holder for lifting, pushing, or pulling. In the present disclosure, however, it is understood that since the front wall 113 includes the cavity 150 that protrudes into the interior space, the cavity 150, having additional space, is configured to provide a comfortable and firm grasping area to facilitate the lifting, pushing, or pulling of the storage container 100. It is appreciated that the disclosure of the handle 155 is not intended to be limiting in scope, but provided to help with the understanding of the disclosure. Other designs of the handle can be considered, for example, as further discussed below in the alternative embodiments.

The lid 120 is substantially planar and provided to enclose the interior space 115 of the housing 110. The lid 120 can be removably attachable and detachable to the housing 110. In an embodiment, the lid 120 is configured to enclose or expose the interior space 115 by a non-destructive and deformable (and restorable) locking and unlocking engagement with the housing 110. As illustrated in FIG. 1C, in an embodiment, the lid 120 can include a rail portion 145 that is configured to engage the rim portion 140 of the side walls 112A, 112B. In an embodiment, the rail portion 145 can include a downwardly open groove that is substantially “C” shaped, “U” shaped, or “V” shaped to engage the rim portion 140 of the side walls 112A, 112B. The rail portion 145 can further include a handhold portion 146 that is connected to the downwardly open groove and extends away from the housing 110 in the width direction that is configured to facilitate the grasping or handling of the rail portion 145 for the non-destructive and deformable detachment or attachment of the lid 120 to the housing 110. It is appreciated that, in an embodiment, since the lid 120 is formed completely of metal, e.g., stainless steel, the deformation of the rail portion 145 can be provided in a non-destructive manner so that the lid can be provided in a locking or unlocking engagement with the rim portion 140 of the side walls 112A, 112B, e.g., due to the deformation and elastic restoration properties of the lid 120. That is, the rail portion 145 is not permanently deformed, e.g., bent (and unbent), for securing the lid 120 to the housing 110, but rather is elastically restored to its original shape. As such, the lid 120 is designed for and can be used for a number of iterative reuses.

In an embodiment, the lid 120 can also be configured to provide a sliding engagement along the rim portion 140 of the housing 110, e.g., via the open groove, to enclose the interior space 115 of the housing 110. In an embodiment, the lid 120 can also be pressed or forced in a downward direction towards the housing 110 such that the rail portion 145, including the handhold portion 146, is configured to engage the rim portion 140, e.g., due to the outwardly extending handhold portion, and elastically deform and then restore, e.g., a snapping action, such that at least one of the groove or the handhold portion 146 of the rail portion 145 is engaged with the rim portion 140 for securement or attachment of the lid 120 to the housing 110.

Referring back to FIG. 1D, in an embodiment, the lid 120 further includes a back rail portion 122 that is configured to engage the back wall 114 of the housing 110. In an embodiment, the back rail portion 122 is configured to align the lid or any openings in any of the rim portion 140 or in the side walls 112A, 112B and the rail portion 145 to enclose the interior space 115.

As such, in an embodiment, the lid 120 is removably attachable and detachable to the housing 110 via a number of different mechanisms to allow the reuse of the storage container 100 after cleaning, sanitizing, or the like. It is also appreciated that, in an embodiment, the lid 120 is not completely attachable and detachable to the housing 110, but rather, the lid 120 can be pivotally connected to the housing 110 such that the rail portion(s) 145 can be non-destructively and deformably locked and unlocked from the rim portion(s) 140 of the housing 110, as further discussed below.

In an embodiment, openings or holes 160 can be provided in the rail portions 145 of the lid 120 and rim portions 140 of the two side walls 112A, 112B. The openings or holes 160 are provided to align the lid 120 with the housing 110 and configured to receive a securing device (not shown) for securing the lid 120 to the housing 110. The securing device can include at least one of a zip tie, securement pin or lock, or a push rivet that allows the securement of the storage container 100 for transport and handling and the removal of which allows the access of the interior space 115 and bag by removing the lid 120.

The first foam portion 130 is configured to conform to a shape of a pharmaceutical or biological materials bag and includes an opening for receiving at least a portion of the pharmaceutical or biological bag, such that the edges of the pharmaceutical or biological bag are fixed within or between the first foam portion 130. The opening of the first foam portion 130 substantially receives the bag portion having the pharmaceutical or biological material and configured such that the first foam portion maintains the shape of the bag as the material in the bag is frozen, e.g., during a plate freezing process in which aqueous solutions expand during freezing, and provides the necessary protection during transport or handling. The first foam portion 130 can include angled edges at one end of the opening to conform to the shape of the pharmaceutical or biological materials bag having angled edges connected to a bottom edge that can include any one of a fitment, connectors, tubing, or the like. In an embodiment, the first foam portion 130 can also include a receiving section nearer the front wall 113 for receiving the bag end having the fitment, connector, or tubing.

The second foam portion 135 is provided adjacent the first foam portion 130 in the interior space and configured to receive any tubing or connections attached to the pharmaceutical or biological materials bag. The second foam portion 135 can include an indentation portion corresponding to the cavity 150. In an embodiment, the second foam portion 135 can also include lateral extension portions that extends towards the back wall of the housing 110 longitudinally along each side wall, such that the entirety or majority of the tubing, connector, or combination thereof, is positioned on or between the second foam portion 135.

The first foam portion 130 and the second foam portion 135 can include corresponding connection portions for providing engagement and securement of the first foam portion 130 and the second foam portion 135, e.g., an insertion portion of the first foam portion 130 between the lateral extension portions of the second foam portion 135, and to provide the support and protection of the bag and the at least one of the fitment, connector, tubing, or the like during the handling, freezing, and transport of the bag.

As illustrated in FIG. 1C, in an embodiment, the first foam portion 130 can include a first portion 131 and a second portion 132 separate from the first portion 131. The first portion 131 of the first foam portion 130 is configured to fit in the first section of the interior space 115 and the second portion 132 is configured to fit in the second section of the interior space 115 above the first portion of the first foam portion 130. The second portion 132 of the first foam portion 130 has a greater volume than the first portion 131 of the first foam portion 130 and is fitted in the interior space 115 including the extension of the housing on the secondary bottom surface 116, e.g., abuts the back wall 114B, while the first portion 131 abuts the back wall 114A. As such, the first portion 131 and the second portion 132 can be used to sandwich or enclose the pharmaceutical or biological material bags between the first portion 131 and the second portion 132 to provide any of the support, protection, conformation of the shape of the pharmaceutical or biological material bag during freezing, transport, or handling.

Similarly, the second foam portion 135 can include a first portion and a second portion that can be mirror images of each other or have different configurations to conform to the interior space 115 of the housing 110. As such, the second foam portion 135 can provide the support and protection of any of the fitment, connector, tubing, or the like connected to the materials bag by having the same sandwiched or provided between the first portion and the second portion.

The first foam portion 130 and the second foam portion 135 can be made of polyurethane, polyethylene, polyvinylidene fluoride, a copolymer thereof, a combination thereof, or the like. It will be appreciated that the material of the first foam portion 130 and the second foam portion 135 can be any suitable material that can tolerate a large range of temperatures (e.g., as low as at or about or below −85° C. without fracturing), be suitable for deforming to adjust to the geometry of the bag (not shown) or components thereof in room temperature, serve as a barrier to help immobilize the bag and serve as a cushion to the bag and components thereof during the freezing, thawing, or the shipping process.

In an embodiment, the second foam portion 135 can be made of a foam that has a lower rigidity than the first foam portion 130. In an embodiment, the second foam portion 135 is made from the same foam material as the first foam portion 130, but has a higher plasticizer content. In an embodiment, the second foam portion 135 is made of a different material than the first foam portion 130. The rigidity level of the second foam portion 135 is chosen such that the second foam portion 135 is able to immobilize and cushion any of the bag fitment or components of the bag, such as, tubing, connector(s), clamp(s), and the like that are more rigid than the bag, e.g., bioprocess bag.

In an embodiment, the housing 110 and lid 120 of the storage container 100 can be made of metal. In an embodiment, the metal can be steel such as stainless steel (e.g., Society of Automotive Engineers (SAE) 304 stainless steel, etc.) or the like. It will be appreciated that the material of the housing 110 and the lid 120 can be any suitable material that provides sufficient robustness, is cost efficient, and provides sufficient thermal conduction. The storage container 100 can be robust enough to handle e.g., being dropped after freezing without the storage container or a bag contained therein experiencing severe damage such as breakage of the storage container 100 or bursting or puncturing of the bag contained therein.

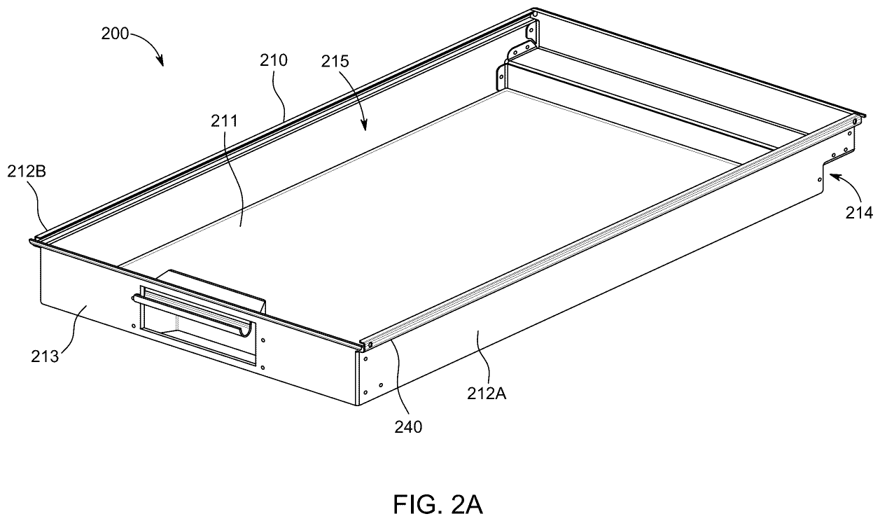

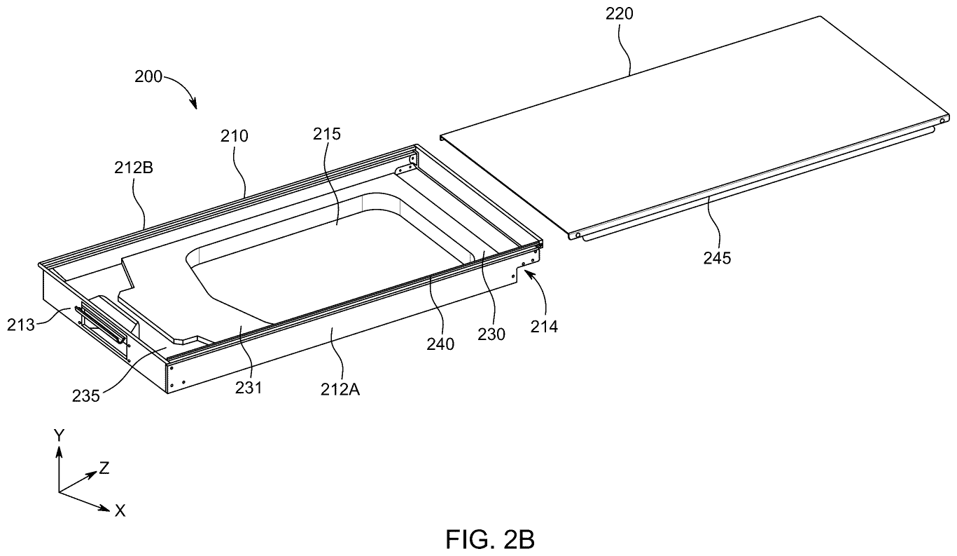

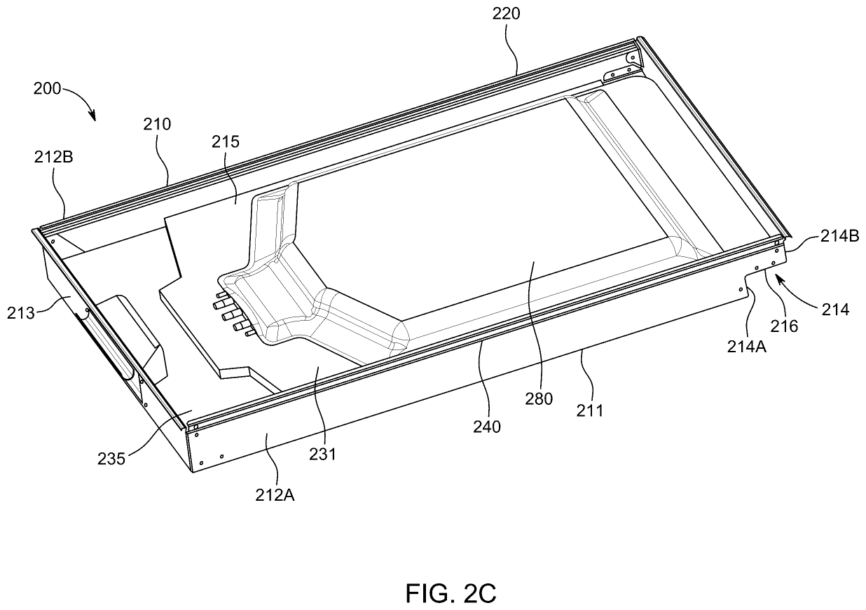

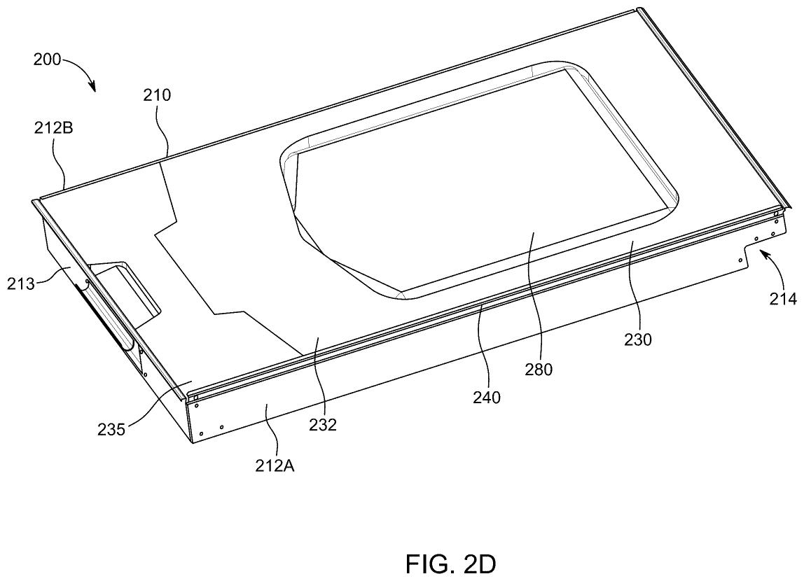

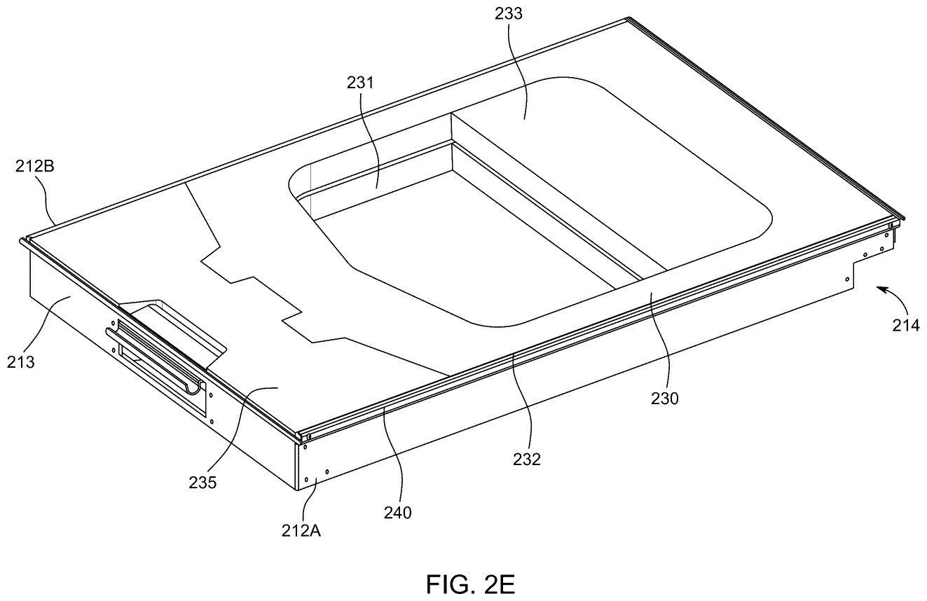

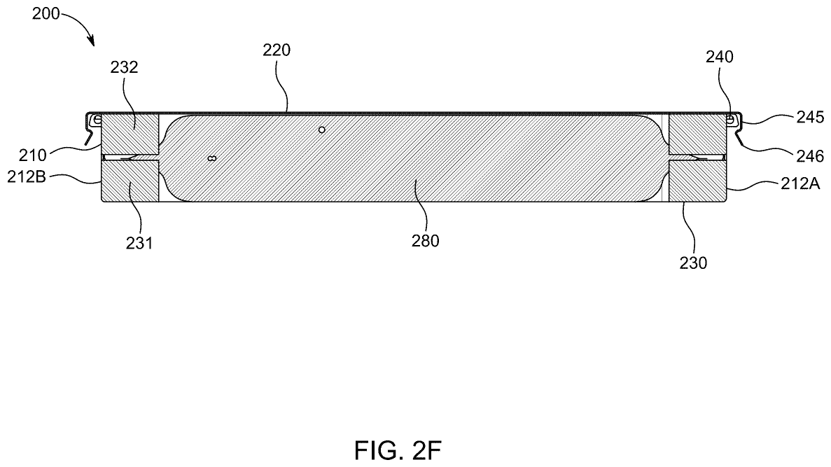

FIGS. 2A to 2F illustrate the assembly of a storage container 200, which can include the same features as the storage container 100 of FIGS. 1A-1F, with the pharmaceutical or biological materials bag, 280 e.g., Entegris Inc.'s Aramus™ bag, according to an embodiment. The storage container 200 provides a greater protection during handling, more consistent freezing/thawing, and reduced storage density, as will be discussed in further detail below.

FIG. 2A illustrates providing a storage container 200 that includes a housing 210. The housing 210 includes a bottom surface 211, two side walls 212A, 212B, a front wall 213, and a back wall 214 that form an interior space 215 in the housing 210. The bottom surface 211, two side walls 212A, 212B, front wall 213, and back wall 214 can be connected together using at least one of self-clinching fasteners, e.g., PEM fasteners, pins, screws, nuts, rivets, or welding at the corners or ends thereof.

As seen in FIG. 2B, when being assembled, in the thickness direction, a first portion 231 of a first foam portion 230 can be disposed or provided in a first section of the interior space 215 above the bottom surface 211 of the housing 210 of the storage container 200. A first portion of a second foam portion 235 can also be provided in the first section of the interior space 215 that is adjacent the first portion of the first foam portion 230, e.g., in the Z-direction.

FIG. 2C further illustrates the storage container 200 when further being assembled, in the thickness direction, e.g., in the Y-direction, in which the pharmaceutical or biological materials bag 280, e.g., the Aramus™ bag, can be disposed or placed above or over the first portion 231 of the first foam portion 230. Any of the tubing can be wrapped in a loose coil and can be disposed or placed above the first portion of the second foam portion 235. Further any clamps on the tubing can be placed in a horizontal orientation to fit in the shell and preferably disposed on the second foam portion 235. In an embodiment, a majority of the tubing and clamps are disposed on the second foam portion 235 and the pharmaceutical or biological materials bag is disposed on the first foam portion 230.

As the storage container 200 is further assembled in FIG. 2D, the second portion 232 of the first foam portion 230 and the second portion of the second foam portion 235 can be disposed or provided above the respective first portions and the pharmaceutical or biological materials bag 280 in a second section of the interior space 215 such that the pharmaceutical or biological materials bag 280 and the tubing and the connectors are between the respective first portions and second portions of the first foam portion 230 and the second foam portion 235.

As illustrated in FIG. 2E, in an embodiment, the first foam portion 230 can include a partial fill foam portion 233 configured to engage the pharmaceutical or biological materials bag (not shown), when the pharmaceutical or biological materials bag is partially filled and inserted into the storage container 200. In prior bags, when the pharmaceutical or biological materials bag is underfilled, the pharmaceutical or biological materials bag does not properly or fully engage the metal surfaces of the storage container 200 for proper freezing of the material, e.g., in a plate freezer. In the present embodiment, however, the partial fill foam portion 233 is configured to displace at least a partial volume in the pharmaceutical or biological materials bag so that the overall volume of the pharmaceutical or biological materials bag is reduced and partial volumes are formed to displace the pharmaceutical or biological materials bag and material therein to ensure proper engagement with the metal surfaces of the storage container 200 for freezing. That is, the partial fill foam portion 233 is configured to raise or increase a height of the partial volumes of the pharmaceutical or biological materials bag for better contact with the metal portions of the storage container 200 and dictates capacity of the bag or portions thereof. It is appreciated that in an embodiment, the partial fill foam portion 233 is not included, but rather, the first foam portion 230 is configured or designed or changeable to reduce the overall volume of the pharmaceutical or biological materials bag, e.g., the shape of the first foam portion 230 can be changed to reduce and displace the volume of material in the pharmaceutical or biological materials bag, e.g., isolates fluid in certain areas of the pharmaceutical or biological materials bag to raise or increase the height of the bag. It is also appreciated that in an embodiment, the lid 220 can also include protrusions or structures that displace the volume of the material in the pharmaceutical or biological materials bag to dictate the capacity of the bag or portions thereof.

As seen in FIGS. 2B and 2C, the back wall 214 of the housing 210 includes a first segment 214A and a second segment 214B provided above the first segment 214A. The second segment 214B is separated a distance longitudinally, with respect to the length of the storage container 200, e.g., Z-direction, from the first segment 214A. The second segment 214B is connected to the first segment 214A by a secondary bottom surface 216. The bottom surface 211 and the first segment 214A of the back wall 214 form a first section of the interior space. The extension of the secondary bottom surface 216 and the second segment 214B form a second section of the interior space that is above the first section and the second section has a greater volume than the first section. It is appreciated that the extended section of the storage container 200 can also be considered a stepped section or ledge section such that the second section has a greater volume than the first section such that the first and second portions of the first foam portion 230 and the second foam portion 235 can be provided.

As seen in FIG. 2F of the final assembled state of the storage container 200, the lid 220 is attached to the housing 210. In an embodiment, the two side walls 212A, 212B include a rim portion 240 at a top portion of the side walls 212A, 212B. In an embodiment, the rim portion 240 can include a bent portion that extends outwardly from the housing and is bent inwardly towards the housing.

The lid 220 can be removably attachable and detachable to the housing 210. In an embodiment, the lid 220 is configured to enclose or expose the interior space 215 by a non-destructive and deformable (and restorable) locking and unlocking engagement with the housing 210. As illustrated in FIG. 2F, in an embodiment, the lid 220 can include a rail portion 245 that is configured to engage the rim portion 240 of the side walls 212A, 212B to enclose the interior space 215. The rail portion 245 can further include a handhold portion 246 that is connected to the downwardly open groove and extends away from the housing 210 in the width direction that is configured to facilitate the grasping or handling of the rail portion 245 for the non-destructive and deformable detachment or attachment of the lid 220 to the housing 210.

In an embodiment, the lid 220 can be attached and detached to the housing 210 in a number of different ways. For example, the lid 220 can be configured to provide a sliding engagement along the rim portion 240 of the housing 210, e.g., via the open groove, to enclose the interior space 215 of the housing 210. In an embodiment, the lid 220 can also be pressed or forced in a downward direction towards the housing 210 such that the rail portion 245, including the handhold portion 246, is configured to engage the rim portion 240, e.g., due to the outwardly extending handhold portion, and elastically deform and then be restored, e.g., a snapping action, such that at least one of the groove or the handhold portion 246 of the rail portion 245 is engaged with the rim portion 240 for securement or attachment of the lid 220 to the housing 210. Similarly, the lid 220 can be detached or removed from the housing 210 by elastically deforming the rail portion 245 of the lid 220 to disengage the rim portion 240 of the housing 210, e.g., by grasping the handhold portion 246, and lifting the lid 220 to expose the interior space 215 such that the lid can be provided in an unlocking engagement without damaging the lid, especially in a clean room environment in which operators are wearing equipment, e.g., gloves, to prevent the contamination of the clean room.

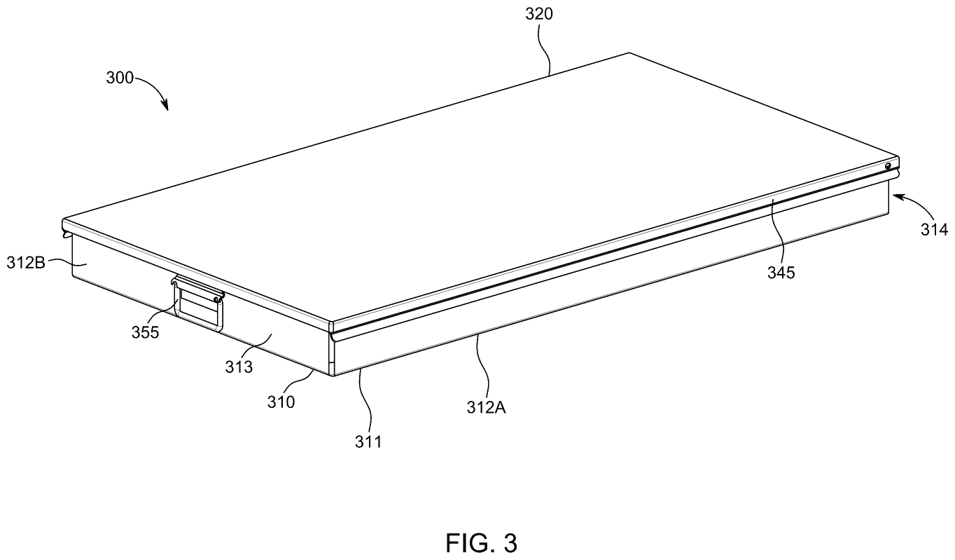

FIG. 3 illustrates a storage container 300, according to an embodiment, which can include the same or similar features as the storage containers 100, 200 of FIGS. 1A-2F, the differences of which are discussed below.

The storage container 300 includes a housing 310, a lid 320, and a first foam portion (e.g., 130, 230) and a second foam portion (e.g., 135, 235). The housing 310 includes a bottom surface 311, two side walls 312A, 312B, a front wall 313, and a back wall 314 that form an interior space in the housing 310.

While the back wall 314 is illustrated as a single wall, it is appreciated that the back wall 314 of the housing 310 can include a first segment and a second segment provided above the first segment, as discussed above with respect to the other embodiments. The second segment is separated a distance longitudinally, with respect to the length of the storage container 300, from the first segment. The second segment is connected to the first segment by a secondary bottom surface. The bottom surface and the first segment of the back wall 314 form a first section of the interior space. The extension of the secondary bottom surface and the second segment form a second section of the interior space that is above the first section and the second section has a greater volume than the first section. It is appreciated that the extended section of the storage container 300 can also be considered a stepped section or ledge section such that the second section has a greater volume than the first section.

The front wall 313 of this embodiment does not include an indentation portion that protrudes into the interior space of the housing 310. Rather, the front wall 313 includes a handle portion 355 configured for lifting, pushing, or pulling the storage container 300. In an embodiment, the handle portion 355 can be a handle that is pivotally connected to the front wall 313. As such, the handle can be extended from the front wall 313 to facilitate the lifting, pushing, or pulling of the storage container 300. It is appreciated that in prior designs of the typical bag holders, the typical bag holders had small openings that made it difficult to hold the bag holder for lifting, pushing, or pulling. In the present disclosure, however, it is understood that since the front wall 313 includes the handle portion 355, the handle portion 355 is configured to provide a comfortable and firm grasping area to facilitate the lifting, pushing, or pulling of the storage container 300.

The lid 320 can be substantially planar and provided to enclose the interior space of the housing 310. The lid 320 can be removably attachable and detachable to the housing 310. In an embodiment, the lid 320 is configured to enclose or expose the interior space by a non-destructive and deformable (and restorable) locking and unlocking engagement with the housing 310. In an embodiment, the lid 320 includes a rail portion 345 that is configured to engage the rim portion (e.g., 140, 240) of the side walls 312A, 312B. It is appreciated that, in an embodiment, since the lid 320 is formed completely of metal, e.g., stainless steel, the deformation of the rail portion 345 can be provided in a non-destructive manner so that the lid can be provided in a locking or unlocking engagement with the rim portion (e.g., 140, 240) of the side walls 312A, 312B, e.g., due to the deformation and elastic restoration properties of the lid 320. That is, the rail portion 345 is not permanently deformed, e.g., bent (and unbent), for securing the lid 320 to the housing 310. As such, the lid 320 is designed for and can be used for a number of iterative reuses.

In an embodiment, the lid 320 can also be configured to provide a sliding engagement along the rim portion (e.g., 140, 240) of the housing 310, e.g., via the open groove, to enclose the interior space of the housing 310. In an embodiment, the lid 320 can also be pressed or forced in a downward direction towards the housing 310 such that the rail portion 345 is configured to engage the rim portion (e.g., 140, 240) e.g., due to the outwardly extending handhold portion, and elastically deform and then restore, e.g., a snapping action, such that the rail portion 345 is engaged with the rim portion (e.g., 140, 240) for securement or attachment of the lid 320 to the housing 310.

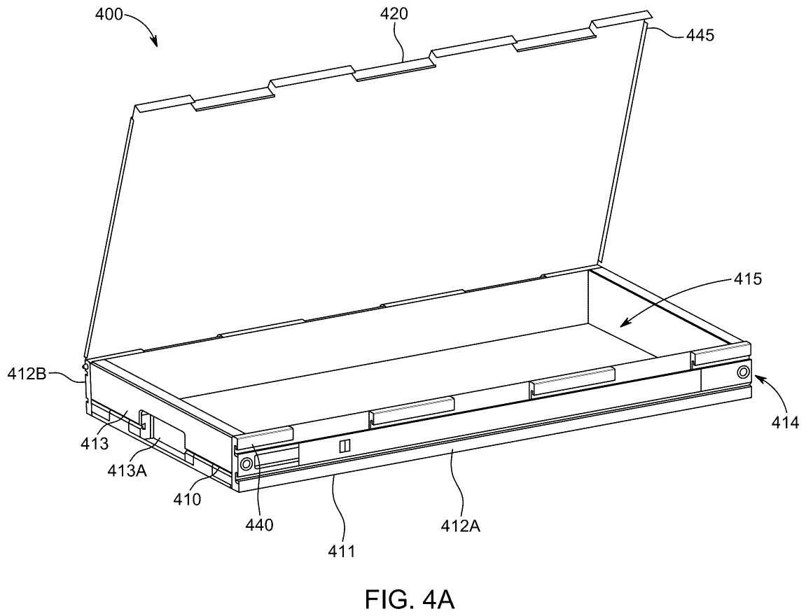

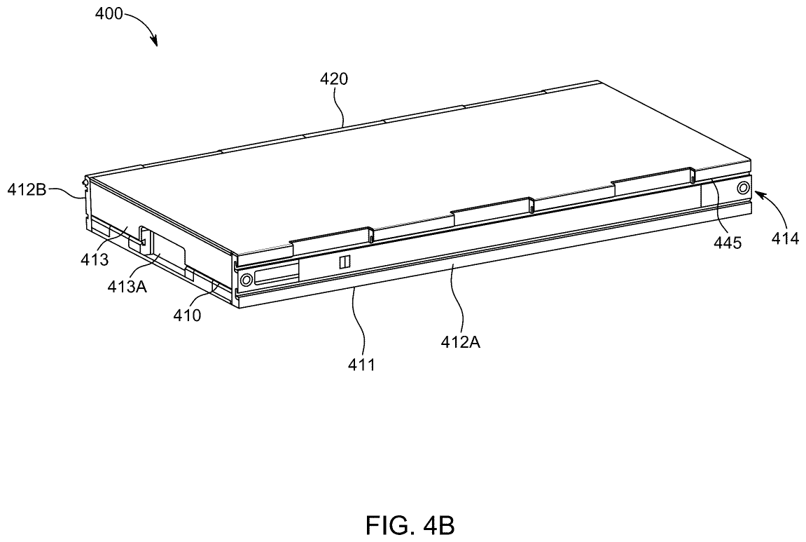

FIGS. 4A and 4B illustrate a storage container 400, according to an embodiment, which can include the same or similar features as any of the storage containers discussed above, the differences of which are discussed below.

The storage container 400 includes a housing 410, a lid 420, and a first foam portion (e.g., 130, 230) and a second foam portion (e.g., 135, 235). The housing 410 includes a bottom surface 411, two side walls 412A, 412B, a front wall 413, and a back wall 414 that form an interior space 415 in the housing 410.

In an embodiment, the lid 420 is pivotally connected to one of the side walls 412A, 412B. For example, in an embodiment, the lid 420 is pivotally connected to the side wall 412B via a living hinge, pin and hinge connection, or the like that allows the pivoting of the lid 420 in an open and closed position to enclose or expose the interior space 415 of the housing 410. The other side wall, e.g., 412A, includes a rim portion 440 at a top portion of the side wall 412A. In an embodiment, the rim portion 440 includes a bent portion or molded portion that extends outwardly from the housing 410.

While the back wall 414 is illustrated as a single wall, it is appreciated that the back wall 414 of the housing 410 can include a first segment and a second segment provided above the first segment, as discussed above with respect to the other embodiments. The second segment is separated a distance longitudinally, with respect to the length of the storage container 400, from the first segment. The second segment is connected to the first segment by a secondary bottom surface. The bottom surface and the first segment of the back wall 414 form a first section of the interior space. The extension of the secondary bottom surface and the second segment form a second section of the interior space that is above the first section and the second section has a greater volume than the first section. It is appreciated that the extended section of the storage container 400 can also be considered a stepped section or ledge section such that the second section has a greater volume than the first section.

The front wall 413 includes an indentation portion 413A that protrudes into the interior space 415 of the housing 410. The indentation portion 413A can be formed from a hole or opening in the front wall 413 and formed as a cavity that protrudes or is positioned within the interior space 415. The cavity can include a handle portion configured for lifting, pushing, or pulling the storage container 400. It is appreciated that the disclosure of the handle is not intended to be limiting in scope, but provided to help with the understanding of the disclosure. Other designs of the handle can be considered, for example, as further discussed above in the alternative embodiments.

The lid 420 can be substantially planar and provided to enclose the interior space 415 of the housing 410. In an embodiment, the lid 420 is configured to enclose or expose the interior space 415 by a non-destructive and deformable (and restorable) locking and unlocking engagement with the housing 410. In an embodiment, the lid 420 includes a rail portion 445 that is configured to engage the rim portion 440 of the side walls 412A. It is appreciated that, in an embodiment, since the lid 420 is formed completely of metal, e.g., stainless steel, or molded plastic, the deformation of the rail portion 445 can be provided in a non-destructive manner so that the lid can be provided in a locking or unlocking engagement with the rim portion 440 of the side walls 412A, e.g., due to the deformation and elastic restoration properties of the lid 420. That is, the rail portion 445 is not permanently deformed, e.g., bent (and unbent), for securing the lid 420 to the housing 410. As such, the lid 420 is designed for and can be used for a number of iterative reuses.

In an embodiment, as seen in FIG. 4A, the rail portion 445 can be provided as staggered portions for the engagement/disengagement with the rim portion 440 which has a corresponding structure to the staggered portions. It is appreciated that the rail portion 445 and rim portion 440 can also be provided a single piece that extends the entirety of the storage container in the length direction, e.g., Z-direction.

In an embodiment, the housing 410 and lid 420 of the storage container 400 can be made of metal. In an embodiment, the metal can be steel such as stainless steel (e.g., Society of Automotive Engineers (SAE) 304 stainless steel, etc.) or the like. It will be appreciated that the material of the housing 410 and the lid 420 can be any suitable material that provides sufficient robustness, is cost efficient, and provides sufficient thermal conduction. The storage container 400 can be robust enough to handle e.g., being dropped after freezing without the storage container or a bag contained therein experiencing severe damage such as breakage of the storage container 400 or bursting or puncturing of the bag contained therein.

In another embodiment, the rim portion 440 can be provided as an insert in the housing 410. For example, the rim portion 440 can be formed from a plastic or molded plastic design that has a second interior space for receiving the foam portions and materials bag and can be configurable to hold different types of materials bags. In another embodiment, the rim portion 440 can be a single piece that is insertable into the side wall of the housing 410. As such, since the rim portion 440 is insertable and removable from the storage container 400, the rim portion 440 can facilitate the use and reuse of the storage container 400, e.g., when the rim portion 440 is damaged, only the rim portion 440 can be replaced or the rim portion 440 can be removed for the cleaning/sanitizing of the storage container 400.

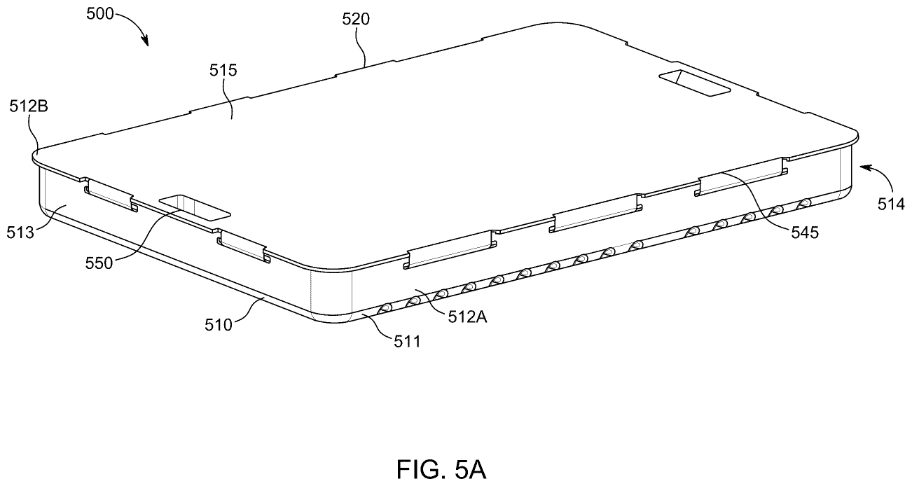

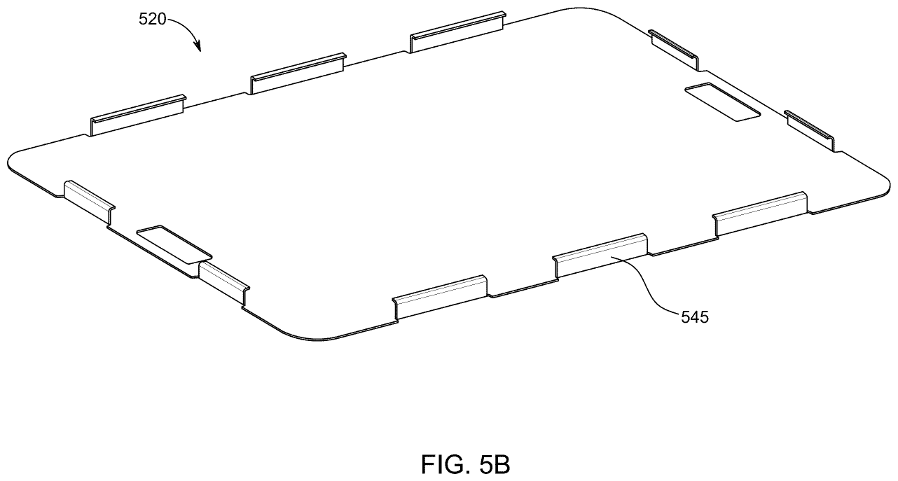

FIGS. 5A and 5B illustrate a storage container 500, according to an embodiment, which can include the same or similar features as any of the storage containers discussed above, the differences of which are discussed below.

The storage container 500 includes a housing 510, a lid 520, and a first foam portion (e.g., 130, 230) and a second foam portion (e.g., 135, 235). The housing 510 includes a bottom surface 511, two side walls 512A, 512B, a front wall 513, and a back wall 514 that form an interior space in the housing 510.

While the back wall 514 is illustrated as a single wall, it is appreciated that the back wall 514 of the housing 510 can include a first segment and a second segment provided above the first segment, as discussed above with respect to the other embodiments. The second segment is separated a distance longitudinally, with respect to the length of the storage container 500, from the first segment. The second segment is connected to the first segment by a secondary bottom surface. The bottom surface and the first segment of the back wall 514 form a first section of the interior space. The extension of the secondary bottom surface and the second segment form a second section of the interior space that is above the first section and the second section has a greater volume than the first section. It is appreciated that the extended section of the storage container 500 can also be considered a stepped section or ledge section such that the second section has a greater volume than the first section.

The front wall 513 of this embodiment does not include an indentation portion that protrudes into the interior space of the housing 510. Rather, a cavity 550 is formed internally in the interior space 515. The cavity 550 can seal the interior space 515 along internal walls (not shown) of the housing 510. The cavity 550 can be provided as a separate element than the storage container 500 or formed during the manufacturing of the storage container 500. If the cavity 550 is provided as a separate element, the cavity 550 can be attached or connected to the storage container 500 using at least one of self-clinching fasteners, e.g., PEM fasteners, pins, screws, nuts, rivets, or welding. The cavity 550 can be formed as a handle portion configured for lifting, pushing, or pulling the storage container 500. It is appreciated that in prior designs of the typical bag holders, the typical bag holders had small openings that made it difficult to hold the bag holder for lifting, pushing, or pulling. In the present disclosure, however, it is understood that since the storage container 500 includes the cavity 550 that is provided in the interior space, the cavity 550, having additional space, is configured to provide a comfortable and firm grasping area to facilitate the lifting, pushing, or pulling of the storage container 500.

The lid 520 can be substantially planar and provided to enclose the interior space 515 of the housing 510. In an embodiment, the lid 520 is configured to enclose or expose the interior space 515 by a non-destructive and deformable (and restorable) locking and unlocking engagement with the housing 510. In an embodiment, the lid 520 includes a rail portion 545 that is configured to engage the rim portion (e.g., 140, 240, 440) of the side walls 512A, 512B. It is appreciated that, in an embodiment, since the lid 520 is formed completely of metal, e.g., stainless steel, or molded plastic, the deformation of the rail portion 545 can be provided in a non-destructive manner so that the lid can be provided in a locking or unlocking engagement with the rim portion of the side walls 512A, 512B, e.g., due to the deformation and elastic restoration properties of the lid 520. That is, the rail portion 545 is not permanently deformed, e.g., bent (and unbent), for securing the lid 520 to the housing 510. As such, the lid 520 is designed for and can be used for a number of iterative reuses. In an embodiment, the lid 520 can include holes or openings in the lid 520 that correspond to the cavities 550 in the interior space 515.

In an embodiment, the lid 520 can also be configured to provide a sliding engagement along the rim portion (e.g., 140, 240, 440) of the housing 510 to enclose the interior space 515 of the housing 510. In an embodiment, the lid 520 can also be pressed or forced in a downward direction towards the housing 510 such that the rail portion 545 is configured to engage the rim portion (e.g., 140, 240, 440), e.g., due to the outwardly extending handhold portion, and elastically deform and then restore, e.g., a snapping action, such that the rail portion 545 is engaged with the rim portion for securement or attachment of the lid 520 to the housing 510.

In an embodiment, the rail portion 545 can be provided as staggered portions for the engagement/disengagement with the rim portion (e.g., 140, 240, 440) which has a corresponding structure to the staggered portions. It is appreciated that the rail portion 545 and rim portion can also be provided a single piece that extends the entirety of the storage container in the length direction, e.g., Z-direction.

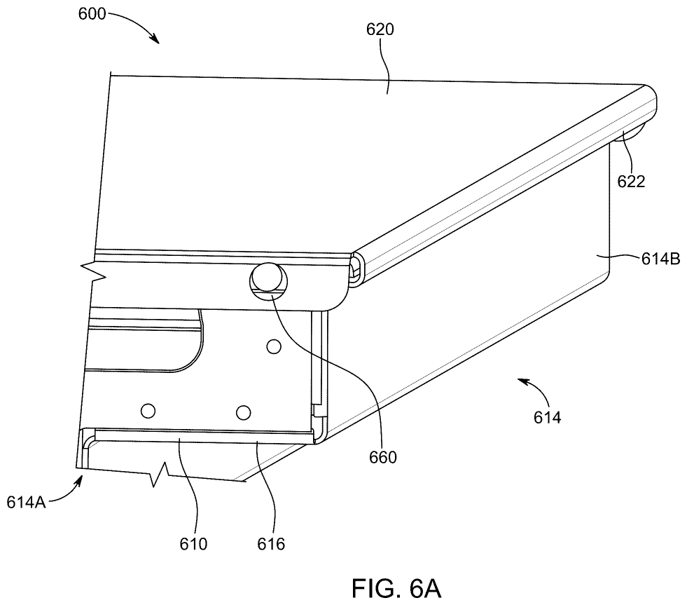

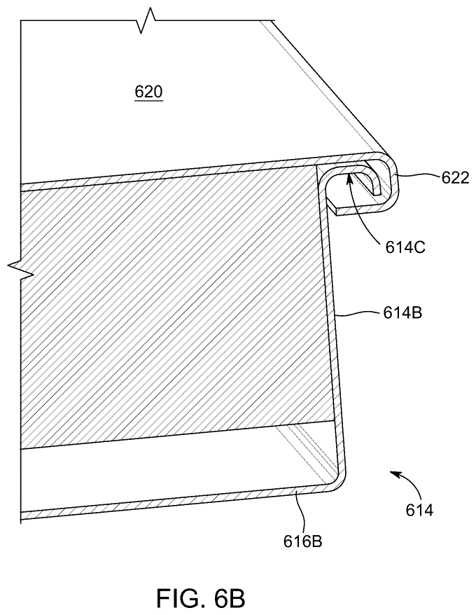

FIGS. 6A and 6B illustrate a storage container 600, according to an embodiment, which can include the same or similar features as any of the storage containers discussed above, the differences of which are discussed below.

As shown in FIG. 6A, a lid 620 includes a back rail portion 622 that is configured as a securing portion adjacent the back wall 614. The back rail portion 622 is configured to engage the back wall 614 to retain and align the lid 620 with the housing 610 of the container 600. In an embodiment, the back rail portion 622 has an open groove that is “C” shaped, “U” shaped, or “V” shaped to engage the back wall 614. The back wall 614 of the housing 610 includes a first segment 614A and a second segment 614B provided above the first segment 614A. The second segment 614B is separated a distance longitudinally, with respect to the length of the storage container 600, from the first segment 614A. The second segment 614B is connected to the first segment by a secondary bottom surface 616.

As seen in FIG. 6B, which is a cross-sectional view of FIG. 6A, the second segment 614B of the back wall 614 includes a lip 614C having a corresponding shape, e.g., C-shaped, for engaging the back rail portion 622 of the lid 620. As such, not only is the lid 620 secured and aligned with the housing 610, such design allows the pivoting of the lid 620 with respect to the housing 610 to have partial attachment thereto, e.g., the open grooves are configured to engage to provide at least a partial attachment.

As such a storage container, and particularly a bag holder, for pharmaceutical or biological material is provided that has a multitude of benefits over the prior designs of the typical bag holders. For example, the storage container can be used for plate freezing and can be used for specific fluoropolymer bags, e.g., Entegris Inc.'s Aramus™ Bag assembly, the storage container is entirely made of sheet metal and foam and has a more cost effective design and cleanability, the storage container has a lid that can either be slid or snapped on/off in a non-destructive and deformable manner and the lid has handhold portions for hand placement and removal of the lid, the foam portions are designed to increase the areas where tubing and connectors are positioned, the storage container does not include any bending of metal or plastic parts which avoids puncture hazards after failure of the part, the handle is enlarged for better gripping, the storage container has a ledge or stepped section to facilitate the assembly of the storage container, the storage container is designed for multiple uses, and the storage container has a more space-efficient design that is compatible for blast freezers. Such a design of the storage container is especially beneficial for use in a clean room environment in which operators wear equipment, e.g., hoods, gloves, or the like, to prevent contamination, e.g., for example, airborne organisms, particles, dust, or the like, of the clean room, which makes it difficult to grasp, hold, manipulate, e.g., open or close, etc., storage containers.

### Aspects:

Any of aspects 1-17 can be combined with any of aspect 18 and aspects 19-21 or vice-versa.

Aspect 1: A storage container for pharmaceutical or biological materials, comprising a housing comprising a bottom surface, two side walls, a front wall, and a back wall that form an interior space in the housing, the interior space having a first section and a second section that is provided above the first section; and a lid configured to enclose the interior space, wherein the two side walls of the housing include a rim portion at a top portion of the two side walls and the lid includes a rail portion configured to provide a non-destructive and deformable locking and unlocking engagement with the rim portion of the two side walls, and the back wall of the housing includes a first segment and a second segment provided above the first segment, the second segment being separated a distance longitudinally from the first segment and connected to the first segment by a secondary bottom surface such that the second section of the interior space has a greater volume than the first section of the interior space.

Aspect 2: The storage container of Aspect 1, wherein the rail portion of the lid is also configured to provide a sliding engagement with the rim portion of the two side walls.

Aspect 3: The storage container of any of Aspects 1-2, wherein the front wall includes an indentation portion such that the indention portion protrudes into the interior space of the housing.

Aspect 4: The storage container of Aspect 3, wherein the indentation portion includes a handle portion configured for lifting, pushing, or pulling the storage container.

Aspect 5: The storage container of any of Aspects 1-4, further comprising a first foam portion provided in the interior space and configured to conform to a shape of a pharmaceutical or biological materials bag; and a second foam portion provided in the interior space adjacent to the first foam portion and configured to receive any tubing or connections attached to the pharmaceutical or biological materials bag, wherein the second foam portion has a lower rigidity than the first foam portion.

Aspect 6: The storage container of any of Aspect 5, wherein the first foam portion includes a first portion and a second portion separate from the first portion, wherein the first portion of the first foam portion is configured to fit in the first section of the interior space and the second portion of the first foam portion is configured to fit in the second section of the interior space above the first portion of the first foam portion.

Aspect 7: The storage container of Aspect 5, wherein the shape of the pharmaceutical or biological materials bag includes angled edges connected to a bottom edge of the pharmaceutical or biological materials bag.

Aspect 8: The storage container of Aspect 5, wherein the first foam portion further includes a partial fill foam portion configured to displace a volume of the pharmaceutical or biological materials bag.

Aspect 9: The storage container of any of Aspects 1-8, wherein the rim portion of the two side walls and the rail portion of the lid include openings configured to align the lid with the housing and configured to receive a securing device for securing the lid to the housing.

Aspect 10: The storage container of Aspect 9, wherein the securing device include at least one of a zip tie, a securement pin or lock, or a push rivet.

Aspect 11: The storage container of Aspect 9, wherein the lid further includes a back rail portion configured to engage the back wall of the housing to align the openings in the rim portion of the two side walls and the rail portion of the lid.

Aspect 12: The storage container of any of Aspects 1-11, wherein the lid has one end pivotally connected to the housing such that the rail portion is configured to provide the non-destructive and deformable locking and unlocking engagement with the rim portion of the two side walls.

Aspect 13: The storage container of any of Aspects 1-11, wherein the lid is removably attachable to the housing.

Aspect 14: The storage container of any of Aspects 1-13, wherein the rail portion of the lid includes a downwardly open groove configured to engage the rim portion of the two side walls.

Aspect 15: The storage container of Aspect 14, wherein the rail portion further includes a handhold portion connected to the downwardly open groove for providing the non-destructive and deformable unlocking engagement between the rim portion and the rail portion to remove the lid from the storage container.

Aspect 16: The storage container of any of Aspects 1-15, wherein the housing and lid are formed completely from metal.

Aspect 17: The storage container of any of Aspects 1-16, wherein the bottom surface, the two side walls, the front wall, and the back wall are connected by at least one of a self-clinching fastener, pin, screw, nut, rivet, or welding.

Aspect 18: A storage container for pharmaceutical or biological materials, comprising a housing comprising a bottom surface, two side walls, a front wall, and a back wall that form an interior space in the housing, the interior space having a first section and a second section that is provided above the first section; a lid configured to enclose the interior space; a first foam portion provided in the interior space and configured to conform to a shape of a pharmaceutical or biological materials bag; and a second foam portion provided in the interior space adjacent to the first foam portion and configured to receive any tubing or connections attached to the pharmaceutical or biological materials bag, wherein the two side walls of the housing include a rim portion at a top portion of the two side walls and the lid includes a rail portion configured to provide a non-destructive and deformable locking and unlocking engagement with the rim portion of the two side walls, the back wall of the housing includes a first segment and a second segment provided above the first segment, the second segment being separated a distance longitudinally from the first segment and connected to the first segment by a secondary bottom surface such that the second section of the interior space has a greater volume than the first section of the interior space, and the front wall includes an indentation portion such that the indention portion protrudes into the interior space of the housing and into the second foam portion, and the indentation portion includes a handle portion configured for lifting, pushing, or pulling the storage container.

Aspect 19: A method for storing a pharmaceutical or biological materials bag comprising providing a first portion of a first foam portion in a first section of an interior space of a housing of a storage container, wherein the housing comprises a bottom surface, two side walls, a front wall, and a back wall that form the interior space in the housing; providing a first portion of a second foam portion in the first section of the interior space adjacent the first portion of the first foam portion; placing the pharmaceutical or biological materials bag over the first portion of the first foam portion and the first portion of the second foam portion such that the pharmaceutical or biological materials bag is provided on the first foam portion and tubing and connectors connected to the pharmaceutical or biological materials bag are provided on the second foam portion; providing a second portion of the first foam portion and a second portion of the second foam portion in a second section of the interior space such that the pharmaceutical or biological materials bag and the tubing and the connectors are between the first portion and the second portion of the first foam portion and the second foam portion, wherein the second section is provided above the first section; and non-destructively deforming a rail portion of the lid to provide a locking engagement of the lid to the housing to enclose the interior space of the housing by engaging the rail portion of the lid with a rim portion at the top portion of the two side walls, wherein the back wall of the housing includes a first segment and a second segment provided above the first segment, the second segment being separated a distance longitudinally from the first segment and connected to the first segment by a secondary bottom surface such that the second section of the interior space has a greater volume than the first section of the interior space.

Aspect 20: The method of Aspect 19, wherein the lid is pivotally connected to the housing such that the lid encloses the interior space by pivoting the lid until the non-destructive deforming of the rail portion of the lid is in the locking engagement with the rim portion of the two side walls.

Aspect 21: The method of Aspect 19, further comprising non-destructively deforming the rail portion of the lid to provide an unlocking engagement of the lid to expose the interior space, without damaging the lid.

The examples disclosed in this application are to be considered in all respects as illustrative and not limitative. The terminology used in this specification is intended to describe particular embodiments and is not intended to be limiting. The terms “a,” “an,” and “the” include the plural forms as well, unless clearly indicated otherwise. The terms “comprises” and/or “comprising,” when used in this specification, specify the presence of the stated features, integers, steps, operations, elements, and/or components, but do not preclude the presence or addition of one or more other features, integers, steps, operations, elements, and/or components. The term “or” as used herein includes any alternatives or combination of alternatives, such as understood by the term and/or, and is not intended as exclusionary of any one of the alternatives.

With regard to the preceding description, it is to be understood that changes may be made in detail, especially in matters of the construction materials employed and the shape, size, and arrangement of parts without departing from the scope of the present disclosure. This specification and the embodiments described are exemplary only, with the true scope and spirit of the disclosure being indicated by the claims that follow.

What is claimed is:

1. A storage container for pharmaceutical or biological materials, comprising:
a housing comprising a bottom surface, two side walls, a front wall, and a back wall that form an interior space in the housing, the interior space having a first section and a second section that is provided above the first section; and
a lid configured to enclose the interior space,
wherein the two side walls of the housing include a rim portion at a top portion of the two side walls and the lid includes a rail portion configured to provide a non-destructive and deformable locking and unlocking engagement with the rim portion of the two side walls, and
the back wall of the housing includes a first segment and a second segment provided above the first segment, the second segment being separated a distance longitudinally from the first segment and connected to the first segment by a secondary bottom surface such that the second section of the interior space has a greater volume than the first section of the interior space.

2. The storage container of claim 1, wherein the rail portion of the lid is also configured to provide a sliding engagement with the rim portion of the two side walls.

3. The storage container of claim 1, wherein the front wall includes an indentation portion such that the indention portion protrudes into the interior space of the housing.

4. The storage container of claim 3, wherein the indentation portion includes a handle portion configured for lifting, pushing, or pulling the storage container.

5. The storage container of claim 1, further comprising:
a first foam portion provided in the interior space and configured to conform to a shape of a pharmaceutical or biological materials bag; and
a second foam portion provided in the interior space adjacent to the first foam portion and configured to receive any tubing or connections attached to the pharmaceutical or biological materials bag,
wherein the second foam portion has a lower rigidity than the first foam portion.

6. The storage container of claim 5, wherein the first foam portion includes a first portion and a second portion separate from the first portion, wherein the first portion of the first foam portion is configured to fit in the first section of the interior space and the second portion of the first foam portion is configured to fit in the second section of the interior space above the first portion of the first foam portion.

7. The storage container of claim 5, wherein the shape of the pharmaceutical or biological materials bag includes angled edges connected to a bottom edge of the pharmaceutical or biological materials bag.

8. The storage container of claim 5, wherein the first foam portion further includes a partial fill foam portion configured to displace a volume of the pharmaceutical or biological materials bag.

9. The storage container of claim 1, wherein the rim portion of the two side walls and the rail portion of the lid include openings configured to align the lid with the housing and configured to receive a securing device for securing the lid to the housing.

10. The storage container of claim 9, wherein the securing device include at least one of a zip tie, a securement pin or lock, or a push rivet.

11. The storage container of claim 9, wherein the lid further includes a back rail portion configured to engage the back wall of the housing to align the openings in the rim portion of the two side walls and the rail portion of the lid.

12. The storage container of claim 1, wherein the lid has one end pivotally connected to the housing such that the rail portion is configured to provide the non-destructive and deformable locking and unlocking engagement with the rim portion of the two side walls.

13. The storage container of claim 1, wherein the lid is removably attachable to the housing.

14. The storage container of claim 1, wherein the rail portion of the lid includes a downwardly open groove configured to engage the rim portion of the two side walls.

15. The storage container of claim 14, wherein the rail portion further includes a handhold portion connected to the downwardly open groove for providing the non-destructive and deformable unlocking engagement between the rim portion and the rail portion to remove the lid from the storage container.

16. The storage container of claim 1, wherein the housing and lid are formed completely from metal.

17. The storage container of claim 1, wherein the bottom surface, the two side walls, the front wall, and the back wall are connected by at least one of a self-clinching fastener, pin, screw, nut, rivet, or welding.

18. A storage container for pharmaceutical or biological materials, comprising:
a housing comprising a bottom surface, two side walls, a front wall, and a back wall that form an interior space in the housing, the interior space having a first section and a second section that is provided above the first section;
a lid configured to enclose the interior space;
a first foam portion provided in the interior space and configured to conform to a shape of a pharmaceutical or biological materials bag; and
a second foam portion provided in the interior space adjacent to the first foam portion and configured to receive any tubing or connections attached to the pharmaceutical or biological materials bag,
wherein the two side walls of the housing include a rim portion at a top portion of the two side walls and the lid includes a rail portion configured to provide a non-destructive and deformable locking and unlocking engagement with the rim portion of the two side walls,
the back wall of the housing includes a first segment and a second segment provided above the first segment, the second segment being separated a distance longitudinally from the first segment and connected to the first segment by a secondary bottom surface such that the second section of the interior space has a greater volume than the first section of the interior space, and
the front wall includes an indentation portion such that the indention portion protrudes into the interior space of the housing and into the second foam portion, and the indentation portion includes a handle portion configured for lifting, pushing, or pulling the storage container.

19. A method for storing a pharmaceutical or biological materials bag comprising:
providing a first portion of a first foam portion in a first section of an interior space of a housing of a storage container, wherein the housing comprises a bottom surface, two side walls, a front wall, and a back wall that form the interior space in the housing;
providing a first portion of a second foam portion in the first section of the interior space adjacent the first portion of the first foam portion;
placing the pharmaceutical or biological materials bag over the first portion of the first foam portion and the first portion of the second foam portion such that the pharmaceutical or biological materials bag is provided on the first foam portion and tubing and connectors connected to the pharmaceutical or biological materials bag are provided on the second foam portion;
providing a second portion of the first foam portion and a second portion of the second foam portion in a second section of the interior space such that the pharmaceutical or biological materials bag and the tubing and the connectors are between the first portion and the second portion of the first foam portion and the second foam portion, wherein the second section is provided above the first section; and
non-destructively deforming a rail portion of the lid to provide a locking engagement of the lid to the housing to enclose the interior space of the housing by engaging the rail portion of the lid with a rim portion at the top portion of the two side walls,
wherein the back wall of the housing includes a first segment and a second segment provided above the first segment, the second segment being separated a distance longitudinally from the first segment and connected to the first segment by a secondary bottom surface such that the second section of the interior space has a greater volume than the first section of the interior space.

20. The method of claim 19, wherein the lid is pivotally connected to the housing such that the lid encloses the interior space by pivoting the lid until the non-destructive deforming of the rail portion of the lid is in the locking engagement with the rim portion of the side walls.

21. The method of claim 19, further comprising non-destructively deforming the rail portion of the lid to provide an unlocking engagement of the lid to expose the interior space, without damaging the lid.
